(19) United States
(12) Patent Application Publication
(10) Pub. No.: US 2004/0010173 A1
(43) Pub. Date: Jan. 15, 2004

(54) Conversion of methane and hydrogen sulfide in non-thermal silent and pulsed corona discharge reactors

(76) Inventors: Pradeep K. Agarwal, Laramie, WY (US); Temi M. Linjewile, Lehi, UT (US)

Correspondence Address: Emery L. Tracy, P.O. Box 1518, Boulder, CO, 80306, US

(21) Appl. No.: 10/393,843

(22) Filed: Mar. 21, 2003

Related U.S. Application Data
(63) Continuation of application No. PCT/US01/30110, filed on Sep. 26, 2001.
(60) Provisional application No. 60/235,998, filed on Sep. 27, 2000.

(51) Int. Cl. C07C 2/02; C07C 4/02; C07C 11/00

(52) U.S. Cl. 585/538; 585/943

(57) ABSTRACT

A method for producing hydrogen from raw feed gases. The method comprises providing a reactor, positioning reactor walls within the reactor, introducing the raw feed gases into the reactor, and reacting the raw feed gases within the reactor to produce hydrogen. An apparatus for the production of hydrogen using a reactor is also provided.

### BACKGROUND OF THE INVENTION

1. Field of the Invention

This invention relates generally to the production of higher C₂ and C₃ hydrocarbons and to the production of elemental sulfur, accompanied by the simultaneous recovery of hydrogen, from feedstreams containing methane and hydrogen sulfide and, more particularly, it describes a new process for the production of acetylene from methane and the production of hydrogen and elemental sulfur from hydrogen sulfide in silent and pulsed corona discharge reactors by continuously recovering hydrogen from the gaseous mixture of products and reactants through a membrane wall.

2. Description of the Prior Art

The principal impetus behind the synthesis of acetylene arises from its value as a chemical intermediate. In the early 1900's, acetylene was used as a raw material in the production of chlorinated solvents, acetic anhydride, and acid, as well as acetone. Starting in 1930's, acetylene was also used as the starting material for a variety of polymers such as synthetic rubbers, vinyl acetate and vinyl chloride monomers required for PVA and PVC, water-base paints, dry-cleaning solvents, and aerosol insecticides.

Two principal routes have been described in the literature for the commercial production of acetylene:

Hydrolysis of calcium carbide formed from the reduction of lime with carbon

Calcium oxide is one of the most stable metal oxides. Production of calcium carbide using the following reaction, then, requires significant expenditure of energy.

$$CaO + 2 C + \frac{1}{2} {O}_{2} -> 2 CO + {CaC}_{2}$$

Not surprisingly, the majority of earlier technological improvements related to the development of the reduction furnace. The hydrolysis reaction:

CaC₂+H₂O→CaO+C₂H₂

is highly exothermic. Temperature control is vital to prevent decomposition of acetylene.

Cracking of hydrocarbons, in particular methane, at high temperatures

More recently, cracking processes for producing acetylene have received significant attention. Methane is used, most often, as the feedstock; other hydrocarbon sources are not available as readily. Several techniques have been described in the literature; however, two key limitations appear to be common to most of these methods. First, acetylene is diluted considerably by reaction products. For example, consider the reaction:

2CH₄→C₂H₂+3H₂

The maximum possible concentration of acetylene, at 100% conversion of methane, is twenty-five (25 vol. %) volume percent. Second, for acetylene production to be favored thermodynamically, the reaction temperature should be greater than about two thousand (2000° K.) degrees Kelvin. At this temperature, conversion to acetylene is rapid; however, sequential decomposition of acetylene to carbon and hydrogen is rapid as well. Clearly, recovery of the acetylene intermediate requires rapid quenching of the product gases. This is difficult in practice since the thermal capacity of gases is low.

The several thermal methods described in the literature for cracking of hydrocarbons to produce acetylene include the following:

Electric arc: This method provides comparatively easy heating of gases to the appropriate reaction temperature. The hot zone, however, can be spatially irregular leading to excessive product decomposition.

Partial oxidation: The raw material is combined with just sufficient oxidizing gas to release the thermal energy required for achieving and maintaining the desired reaction temperature. Quenching of gases remains difficult though product dilution can be minimized by use of oxygen.

Regenerative pyrolysis: In this method, a structure of refractory shapes is heated through intermittent flow of oxidizing gas. In between the periods corresponding to oxidizing gas flow, hydrocarbons contact the heated surfaces and undergo endothermic pyrolytic cracking.

Submerged flame: A flame is propagated in within the bulk of a liquid hydrocarbon. The high temperature required for reaction is achieved in the flame region. Quenching is rapid.

Other thermal methods—for example, triboelectric discharge and laser irradiation—have also been described more recently in the patent literature. Expensive and potentially corrosive reaction chamber is necessary for laser irradiation; and triboelectric discharge involves potentially dangerous pressure changes.

Non-thermal discharges have attempted to overcome the shortcomings of thermal methods. Such non-equilibrium plasmas have been divided into five distinctive groups depending on the mechanism used for their generation, applicable pressure range, and electrode geometry. These are as follows:

Glow discharge: This is an essentially low-pressure phenomenon usually between flat electrodes. The low pressure and mass flow severely restrict chemical industrial application.

Corona Discharge: Use of inhomogeneous electrode geometries permits stabilization of discharges at high pressure. Several specific regions of operation—for example, ac or dc, and pulsed—have been described in the literature for applications involving, most often, cleanup of flue gas and atmospheric pollutants. The use of dc corona discharges for the production of acetylene from methane has been described. The AC/DC corona discharges, however, are inefficient in their higher energy consumption. The use of pulsed corona discharges for the production of acetylene from methane is one of the embodiments of the present patent application.

Silent Discharge: In this operational regime, one or both of the electrodes are covered with a dielectric layer. Application of a sinusoidal (or other time-varying) voltage, then, leads to pulsing electric fields and microdischarges similar to those observed in pulsed corona discharge systems.

RF Discharge: In such systems, the electrodes are not an integral part of the discharge volume. Non-thermal (or non-equilibrium) conditions are expected only at low pressures whereas thermal plasmas, with the limitations discussed earlier, can be expected at high pressures—and larger production rates—of interest in the chemical process industry.

Microwave Discharge: Here, similar to RF discharge systems, the electrodes are not an integral part of the discharge volume. The wavelength of the applied electromagnetic field becomes comparable to the dimensions of the discharge volume and necessitates other coupling mechanisms. Several patents have been issued on the use of microwave energy for the production of acetylene from methane. Used metal/non-metal composites (elongated structural construction) within the discharge volume and a pulsating microwave energy source have been described. Using similar internals in the discharge volume but with a continuous microwave energy source has also been described. Other catalytic materials have also been used within the discharge volume. The use of activated charcoal as catalyst/reactant within the discharge volume has been described. The use of catalytic pellets within the discharge volume can lead to deposition of carbon on the internal surfaces and, therefore, intermittent operation. Others have, consequently, generated plasma using microwave energy; this plasma was introduced into a reactor loaded with catalyst.

In comparing these non-thermal plasmas, it must be noted that in a glow discharge, the electrons gain energy from the applied field. Due to low pressures, collision with neutral species is infrequent. Propensity for the creation of reactive ions and chemical species is limited. Steady state operation is governed, primarily, by loss of energy incurred by the electrons on enclosure walls and other surfaces within the reactor. The situation is similar in RF and microwave discharges. In corona and silent discharges, the situation is entirely different; these are the operating regimes that are embodied in the present patent application. The fast electrons do indeed transfer energy to other molecules in the system. Electrode geometry and construction prevent sparking or arcing. Propensity for the creation of reactive ions and chemical species is very high.

### SUMMARY

The present invention is a method for the production of acetylene. The method comprises providing raw feed gases consisting of methane, introducing the raw feed gases into a reactor, positioning reactor walls within the reactor, and reacting the raw feed gases within the reactor with the following reaction:

2CH₄→C₂H₂+3H₂.

The present invention additionally including an apparatus for the production of acetylene. The apparatus comprises raw feed gases consisting of methane, a reactor for reacting the raw feed gases within the reactor, and reactor walls positioned within the reactor wherein the following reaction occurs:

2CH₄→C₂H₂+3H₂.

The present invention further includes a method for producing hydrogen from raw feed gases. The method comprises providing a reactor, positioning reactor walls within the reactor, introducing the raw feed gases into the reactor, and reacting the raw feed gases within the reactor to produce hydrogen.

The present invention further still includes a method for the production of hydrogen and elemental sulfur. The method comprises providing raw feed gases consisting of hydrogen sulfide (H₂S), introducing the raw feed gases into a reactor, positioning reactor walls within the reactor, and reacting the raw feed gases within the reactor with at least one of the following reactions:

H₂S→H+SH

H+SH→2H+S

2H→H₂

H₂S+H→SH+H₂.

The present invention further yet includes an apparatus for the production of hydrogen and elemental sulfur. The apparatus comprises raw feed gases consisting of hydrogen sulfide (H₂S), a reactor for reacting the raw feed gases within the reactor, and reactor walls positioned within the reactor wherein at least one of the following reactions occur:

H₂S→H+SH

H+SH→2H+S

2H→H₂

H₂S+H→SH+H₂.

### BRIEF DESCRIPTION OF THE DRAWINGS

FIG. 1 is a schematic view of the apparatus and method for the conversion of methane in non-thermal silent and pulsed corona discharge reactors, constructed in accordance with the present invention; and

FIG. 2 is a schematic view of the apparatus and method for the conversion of hydrogen sulfide in non-thermal silent and pulsed corona discharge reactors, constructed in accordance with the present invention.

### DETAILED DESCRIPTION OF THE PREFERRED EMBODIMENTS

The present invention concerns utilizing either a non-thermal pulsed plasma corona reactor or a silent barrier reactor having membranes positioned therein and receiving co-axial or other gas flow patterns. The present invention permits collection of purified hydrogen and provides significant energy and conversion advantages.

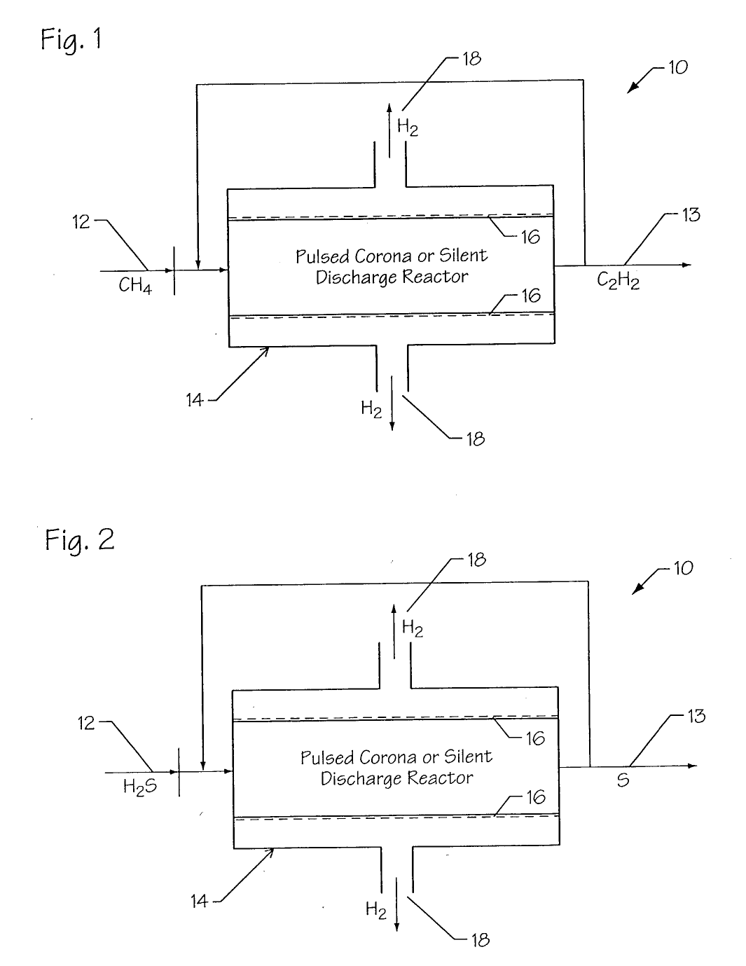

As illustrated in FIG. 1, the present invention is an apparatus and method, indicated generally at 10, for the production of acetylene 11 (and other C₂ and C₃ hydrocarbons), using methane as a raw feed gas 12, and for the production of elemental sulfur and hydrogen using hydrogen sulfide (H₂S) as a raw feed gas 12, both in a silent discharge and non-thermal pulsed plasma corona reactor 14. It should be noted that the present invention can utilize either a silent discharge reactor or a non-thermal pulsed corona reactor.

The raw feed gas 12 is available in sour natural gas streams and the production facility for producing the acetylene 11 and the hydrogen and elemental sulfur can be sited, to advantage, near such gas fields. The principal overall reaction for production of the acetylene 11 within the non-thermal pulsed plasma corona reactor 14 is as follows:

2CH₄→C₂H₂+3H₂ (A)

Within the non-thermal pulsed plasma corona reactor 14, conversion is expected to proceed through the dissociation of methane and hydrogen sulfide by energetic electrons according to the following:

CH₄→CH₃+H (B)

CH₃→CH₂+H (C)

CH₂→CH+H (D)

CH→C+H (E)

The recombination of the radical species leads to the following:

CH₃+CH₃→C₂H₆ (F)

CH₂+CH₂→C₂H₄ (G)

CH+CH→C₂H₂ (H)

H+H→H₂ (I)

High voltage pulses in the non-thermal pulsed plasma corona reactor 14 produce short-lived microdischarges that preferentially accelerate the electrons without imparting significant energy to the ions. The high voltage pulses within the non-thermal pulsed plasma corona reactor 14 lowers power consumption. In addition, most of the energy applied goes to accelerating the electrons rather than the comparatively massive ions. Larger reactor volumes are consequently possible.

The non-thermal pulsed plasma corona reactor 14 has reactor walls 16 constructed from membrane materials—for example, palladium coated substrates, carbon among others—which permit selective permeation of hydrogen 18. Continuous removal of hydrogen 18 through the reactor walls 16 pushes reaction A towards completion. The membrane materials can be coated with a corrosive resistant material such as platinum or the like.

A schematic diagram illustrating the apparatus and method of the present invention is illustrated in FIG. 1. It should be noted, however, that alternative arrangements devised to exploit the process concept more advantageously are within the scope of this invention.

As illustrated in FIG. 2, and as described above, the present invention further includes the conversion of hydrogen sulfide 13 to elemental sulfur 13 and hydrogen 18 in a non-thermal pulsed corona reactor 14. The H₂S, CO₂, and CH₄ from a regenerator (not shown) will form the primary feed to the non-thermal pulsed corona reactor 14. Recovery of elemental sulfur 22 and hydrogen 18 from H₂S in the non-thermal pulsed corona reactor 14 is based, primarily, on the following reactions:

H₂S→H+SH (6)

H+SH→2H+S (7)

2H→H₂ (8)

H₂S+H→SH+H₂ (9)

The emphasis is on the dissociation of H₂S according to Reaction (6). Formation of sulfur occurs by Reaction (7). Reactions (8) and (9) are responsible for the formation of hydrogen. Since the feed gas stream to the non-thermal pulsed corona reactor 14 consists of H₂S and CO₂, the following reaction can also take place:

H₂S+CO₂→H₂O+CO+S (10).

The approach herein has a distinct advantage in that the fuel value of H₂S is transformed to CO and H₂; this synthesis gas can actually be burnt to meet the energy requirements of the process. While CO₂ also leads to the formation of COS, its production can be minimized by choice of proper operating conditions.

The reactions and processes described herein can also be viewed as a substitute for the Claus chemistry and operations used widely for sulfur recovery from streams containing hydrogen sulfide.

The advantages of the apparatus and process 10 of the present invention are clear:

The present invention permits the production of acetylene (and other C₂ and C₃ hydrocarbons) 11 and elemental sulfur 22 and hydrogen 18 from relatively inexpensive feedstock. Expensive preheating and pressurization of the feed gases 12 is also not required. The hydrogen 18 separation is relatively simple.

The present invention permits simultaneous production of hydrogen 18. The fuel value of methane is recovered in the form of cleaner-burning hydrogen. The hydrogen 14 can find use within the petroleum refinery if the process is used in conjunction with a desulfurization unit. Alternatively, hydrogen 14 can be used to generate clean electricity using fuel-cell technology.

The present invention can be utilized for methane, hydrogen sulfide, or mixtures thereof, along with other gases. The products, besides the hydrogen, will vary with operating conditions and feed mixture composition. Also, the present invention can be integrated readily into fuel cell applications.

The foregoing exemplary descriptions and the illustrative preferred embodiments of the present invention have been explained in the drawings and described in detail, with varying modifications and alternative embodiments being taught. While the invention has been so shown, described and illustrated, it should be understood by those skilled in the art that equivalent changes in form and detail may be made therein without departing from the true spirit and scope of the invention, and that the scope of the present invention is to be limited only to the claims except as precluded by the prior art. Moreover, the invention as disclosed herein, may be suitably practiced in the absence of the specific elements which are disclosed herein.

What is claimed is:

1. A method for the production of acetylene, the method comprising:
providing raw feed gases consisting of methane;
introducing the raw feed gases into a reactor;
positioning reactor walls within the reactor; and
reacting the raw feed gases within the reactor with the following reaction:
2CH₄→C₂H₂+3H₂.

2. The method of claim 1 wherein the reactor is selected from the group consisting of a non-thermal pulsed plasma corona and a silent discharge reactor.

3. The method of claim 1 wherein the raw feed gases are collected from sour natural gas streams.

4. The method of claim 1 wherein the reaction within the reactor proceeds through the dissociation of methane by energetic electrons according to the following reactions:
CH₄→CH₃+H
CH₃→CH₂+H
CH₂→CH+H
CH→C+H.

5. The method of claim 4 wherein the recombination of the radical species proceeds according to the following reactions:
CH₃+CH₃→C₂H₆
CH₂+CH₂→C₂H₄
CH+CH→C₂H₂
H+H→H₂.

6. The method of claim 1 and further comprising:
high voltage pulses in the reactor, the high voltage pulses producing short-lived microdischarges that accelerate the electrons without imparting significant energy to the ions.

7. The method of claim 1 wherein the reactor walls are constructed from membrane materials, the membrane materials allowing selective permeation of hydrogen for continuous removal of hydrogen through the membrane materials.

8. The method of claim 7 wherein the membrane materials are selected from the group consisting of palladium coated substrates and carbon.

9. The method of claim 8 and further comprising:
coating the membrane materials with a corrosive resistant material.

10. The method of claim 8 wherein the corrosive resistant material is constructed from a platinum material.

11. An apparatus for the production of acetylene, the apparatus comprising:
raw feed gases consisting of methane;
a reactor for reacting the raw feed gases within the reactor; and
reactor walls positioned within the reactor;
wherein the following reaction occurs:
2CH₄→C₂H₂+3H₂.

12. The apparatus of claim 11 wherein the reactor is selected from the group consisting of a non-thermal pulsed plasma corona and a silent discharge reactor.

13. The apparatus of claim 11 wherein the raw feed gases are collected from sour natural gas streams.

14. The apparatus of claim 11 wherein the reaction within the reactor proceeds through the dissociation of methane by energetic electrons according to the following reactions:
CH₄→CH₃+H
CH₃→CH₂+H
CH₂→CH+H
CH→C+H.

15. The apparatus of claim 14 wherein the recombination of the radical species proceeds according to the following reactions:
CH₃+CH₃→C₂H₆
CH₂+CH₂→C₂H₄
CH+CH→C₂H₂
H+H→H₂.

16. The apparatus of claim 11 wherein the reactor includes high voltage pulses, the high voltage pulses producing short-lived microdischarges that accelerate the electrons without imparting significant energy to the ions.

17. The apparatus of claim 11 wherein the reactor walls are constructed from membrane materials, the membrane materials allowing selective permeation of hydrogen for continuous removal of hydrogen through the membrane materials.

18. The apparatus of claim 17 wherein the membrane materials are selected from the group consisting of palladium coated substrates and carbon among others.

19. The apparatus of claim 18 and further comprising:
coating the membrane materials with a corrosive resistant material.

20. The apparatus of claim 19 wherein the corrosive resistant material is constructed from a platinum material.

21. A method for producing hydrogen from raw feed gases, the method comprising:
providing a reactor;
positioning reactor walls within the reactor;
introducing the raw feed gases into the reactor; and
reacting the raw feed gases within the reactor to produce hydrogen.

22. The method of claim 21 wherein the reactor is selected from the group consisting of a non-thermal pulsed plasma corona and a silent discharge reactor.

23. The method of claim 21 wherein the raw feed gases are collected from sour natural gas streams.

24. The method of claim 21 wherein the raw feed gases consist of methane and hydrogen sulfide are reacted within the non-thermal pulsed plasma corona reactor with the following reaction:
CH₄+H₂S→CH₃SH+H₂
to produce hydrogen.

25. The method of claim 24 wherein the reaction within the non-thermal pulsed plasma corona reactor proceeds through the dissociation of methane by energetic electrons according to the following reactions:
CH₄→CH₃+H
CH₃→CH₂+H
CH₂→CH+H
CH→C+H.

26. The method of claim 25 wherein the recombination of the radical species proceeds according to the following reactions:
CH₃+CH₃→C₂H₆
CH₂+CH₂→C₂H₄
CH+CH→C₂H₂
H+H→H₂.

27. The method of claim 21 wherein the raw feed gases consist of hydrogen sulfide (H₂S) are reacted within the reactor with one of the following reactions:
H₂S→H+SH
H+SH→2H+S
2H→H₂
H₂S+H→SH+H₂.
to produce hydrogen.

28. The method of claim 27 wherein the reaction within the reactor proceeds through the dissociation of hydrogen sulfide by energetic electrons according to the following reaction:
H₂S+CO₂→H₂O+CO+S.

29. The method of claim 21 and further comprising:
high voltage pulses in the reactor, the high voltage pulses producing short-lived microdischarges that accelerate the electrons without imparting significant energy to the ions.

30. The method of claim 21 wherein the reactor walls are constructed from membrane materials, the membrane materials allowing selective permeation of hydrogen for continuous removal of hydrogen through the membrane materials.

31. The method of claim 27 wherein the membrane materials are selected from the group consisting of palladium coated substrates and carbon among others.

32. The method of claim 31 and further comprising:
coating the membrane materials with a corrosive resistant material.

33. The method of claim 32 wherein the corrosive resistant material is constructed from a platinum material.

34. A method for the production of hydrogen and elemental sulfur, the method comprising:
providing raw feed gases consisting of hydrogen sulfide (H₂S);
introducing the raw feed gases into a reactor;
positioning reactor walls within the corona reactor; and
reacting the raw feed gases within the reactor with at least one of the following reactions:
H₂S→H+SH
H+SH→2H+S
2H→H₂
H₂S+H→SH+H₂.

35. The method of claim 34 wherein the reactor is selected from the group consisting of a non-thermal pulsed plasma corona and a silent discharge reactor.

36. The method of claim 34 wherein the raw feed gases are collected from sour natural gas streams.

37. The method of claim 34 wherein the reaction within the reactor proceeds through the dissociation of hydrogen sulfide by energetic electrons according to the following reaction:
H₂S+CO₂→H₂O+CO+S.

38. The method of claim 34 and further comprising:
high voltage pulses in the reactor, the high voltage pulses producing short-lived microdischarges that accelerate the electrons without imparting significant energy to the ions.

39. The method of claim 34 wherein the reactor walls are constructed from membrane materials, the membrane materials allowing selective permeation of hydrogen for continuous removal of hydrogen through the membrane materials.

40. The method of claim 39 wherein the membrane materials are selected from the group consisting of palladium coated substrates and carbon.

41. The method of claim 40 and further comprising:
coating the membrane materials with a corrosive resistant material.

42. The method of claim 41 wherein the corrosive resistant material is constructed from a platinum material.

43. An apparatus for the production of hydrogen and elemental sulfur, the apparatus comprising:
raw feed gases consisting of hydrogen sulfide (H₂S);
a reactor for reacting the raw feed gases within the reactor; and
reactor walls positioned within the reactor;
wherein at least one of the following reactions occur:
H₂S→H+SH
H+SH→2H+S
2H→H₂
H₂S+H→SH+H₂.

44. The apparatus of claim 43 wherein the reactor is selected from the group consisting of a non-thermal pulsed plasma corona and a silent discharge reactor.

45. The apparatus of claim 43 wherein the raw feed gases are collected from sour natural gas streams.

46. The apparatus of claim 43 wherein the reaction within the reactor proceeds through the dissociation of hydrogen sulfide by energetic electrons according to the following reactions:
H₂S+CO₂→H₂O+CO+S.

47. The apparatus of claim 43 wherein the reactor includes high voltage pulses, the high voltage pulses producing short-lived microdischarges that accelerate the electrons without imparting significant energy to the ions.

48. The apparatus of claim 43 wherein the reactor walls are constructed from membrane materials, the membrane materials allowing selective permeation of hydrogen for continuous removal of hydrogen through the membrane materials.

49. The apparatus of claim 48 wherein the membrane materials are selected from the group consisting of palladium coated substrates and carbon among others.

50. The apparatus of claim 49 and further comprising:
coating the membrane materials with a corrosive resistant material.

51. The apparatus of claim 50 wherein the corrosive resistant material is constructed from a platinum material.
